(19) United States
(12) Patent Application Publication
(10) Pub. No.: US 2023/0355871 A1
(43) Pub. Date: Nov. 9, 2023

(54) Minimally Invasive and Semi-Automated Myocardial Injection Device

(71) Applicants: University of South Carolina, Columbia, SC (US); THE UNITED STATES GOVERNMENT AS REPRESENTED BY THE DEPARTMENT OF VETERANS AFFAIRS, Washington, DC (US)

(72) Inventors: Dara Ahmadi Azar, Brookline, MA (US); Tarek M. Shazly, Columbia, SC (US); Francis G. Spinale, Blythewood, SC (US)

(73) Assignee: University of South Carolina, Columbia, SC (US)

(21) Appl. No.: 18/245,191

(22) PCT Filed: Sep. 14, 2021
(86) PCT No.: PCT/US2021/050184

§ 371 Date: Mar. 14, 2023

Related U.S. Application Data
(60) Provisional application No. 63/078,458, filed on Sep. 15, 2020.

(51) Int. Cl. A61M 5/158

(52) U.S. Cl. CPC A61M 5/158; A61M 2210/125; A61M 2230/65; A61M 2205/3306; A61M 2005/1588

(57) ABSTRACT

Described herein are devices and methods for performing automated and minimally invasive intramyocardial injections for cardiac repair that eliminate the need for opening the chest cavity for injections of therapeutics to the heart muscle to address heart attack, cardiomyopathy or myocardial diseases and can detect diseased tissue and deliver a specified volume of a therapeutic injectate to the region of interest.

### TECHNICAL FIELD

The subject matter disclosed herein is generally directed to devices and methods for performing automated and minimally invasive intramyocardial injections for cardiac repair that eliminate the need for opening the chest cavity for injections of therapeutics to the heart muscle to address heart attack, cardiomyopathy or myocardial diseases and can detect diseased tissue and deliver a specified volume of a therapeutic injectate to the region of interest.

### BACKGROUND

Approximately, 550,000 first episodes and 200,000 recurrent episodes of acute myocardial infarction occur annually. Currently, intramyocardial injection therapy is performed by means of invasive surgery to gain access to the diseased (targeted) section of the heart wall.

Accordingly, it is an object of the present disclosure to provide much less invasive devices and methods for use in cardiac repair to deliver therapeutic injectates to the infarct heart without the need of open chest surgery. Additionally, a semi-automated delivery platform will reduce the procedural risks around engaging with an on-beat organ and increase the overall impact of innovation in various therapeutics areas related to the heart.

Citation or identification of any document in this application is not an admission that such a document is available as prior art to the present disclosure.

### SUMMARY

The above objectives are accomplished according to the present disclosure by providing in one embodiment, a method for delivering therapeutic injectates to a heart. The method may include introducing at least one intrusion needle through a chest wall, positioning the at least one intrusion needle adjacent the heart of a treatment subject via use of at least one cardiac mapping technique, inserting at least one probing device through the at least one intrusion needle to locate at least one injection site on a myocardium of the treatment subject, introducing at least one secondary needle for intrusion into the myocardium; and injecting a therapeutic injectate via the at least one secondary needle when movement of the heart of the treatment subject causes the heart to engage the at least one secondary needle and delivery of the therapeutic agent occurs without performing thoracotomy while the heart is on-beat. Further, the at least one probing device may be a fiber angioscope producing real time images. Still, the at least one probing device may be a conductive probe that measures bio-impedance of the myocardium. Yet still, the secondary needle may engage the myocardium orthogonally. Still again, positioning of the secondary needle may be adjusted via a control system that can position the secondary needle prior to, during, and after an injection into the myocardium. Furthermore, the injection occurs when the heart of the treatment subject is enlarged during the cardiac cycle. Yet further, an injection control module may determine time points for injection, volume to deliver, initiation, and stoppage of the injection. Again further, a hydrodynamic system may employ a precision pump to control injectate delivery. Moreover, the at least one injection may occur at an infarct area of the heart.

In a further embodiment, a semi-automated delivery platform for reducing invasive heart operations is provided. The platform may include at least one intrusion needle, at least one control system, at least one delivery apparatus, at least one therapeutic agent, at least one probing device, at least one injecting device wherein movement of a heart of the treatment subject may cause the heart to engage the at least one injecting device; and delivery of the therapeutic agent may occur without performing thoracotomy and while the heart is on-beat. Further, the at least one probing device may be a fiber angioscope producing real time images. Still, the at least one probing device may be a conductive probe that measures bio-impedance of the myocardium. Again, the injecting device engages the myocardium orthogonally. Moreover, positioning of the injecting device may be adjusted via a control system that can position the secondary needle prior to, during, and after an injection into the myocardium. Still further, the injection may be timed to occur when the heart of the treatment subject is enlarged during the cardiac cycle. Again, an injection control module determines time points for injection, volume to deliver, initiation, and stoppage of the injection. Still moreover, a hydrodynamic system employing a precision pump may control injectate delivery. Even further, the at least one injection may occur at an infarct area of the heart.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows a diagram of one embodiment of a targeted intramyocardial injection.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transderm al, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the compounds and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration of a pharmaceutical formulation thereof such as any of the methods described in greater detail elsewhere herein.

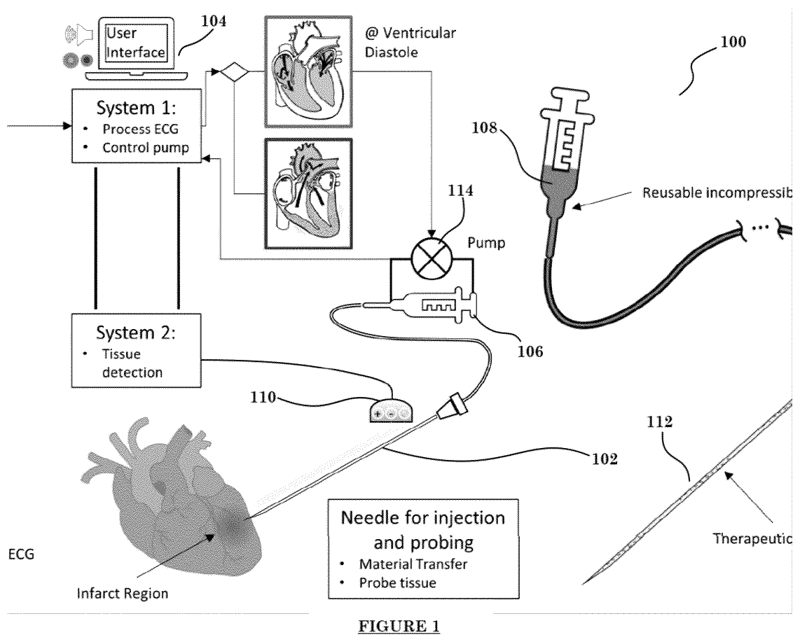

The disclosed medical device is conceptualized, designed and currently is under further development to perform controlled intramyocardial injections in a manner that does not require invasive surgery by utilizing the transepicardial route of direct administration. The device 100, see FIG. 1, comprises a multipurpose needle 102, control systems 104, delivery apparatus 106 and a therapeutic agent 108.

Instead of performing thoracotomy (opening of the chest) to access the myocardium, our technology allows for direct and semi-automated injection using an intrusion needle that is modified for this purpose and will be used as an access point for probing and injectate delivery. The intrusion needle will be guided through the chest wall and positioned near the beating heart of the study/treatment subject. This initial placement can be supported by ultrasonic visualization of the heart in the chest cavity, fluoroscopy, or other real-time cardiac mapping techniques. After insertion of the intrusion needle (primary needle) a probing device 110 can be inserted through the primary needle to detect/locate the site of injection on the myocardium. Probing device or module can be a fiber angioscope that produces real-time images that can be analyzed by the operator or the controller software; or it may be a conductive probe that measures bio-impedance of the myocardium. Control software may include software used to “drive or control” the motors and the pumping of medication in an infusion pump; or software used in closed loop control used in hardware medical devices. This includes “embedded software”, “firmware”, or “micro-code” as known to those of skill in the art. This software may also be software that encrypts data for transmission from the probing device 110. The software controls may also be software that monitors performance or proper functioning of the devices for the purpose of service or software that integrates and analyzes laboratory quality control data to identify increased random errors or trends in calibration.

After identification of the site of injection, a secondary needle 112 (smaller than the primary needle, may be guided through the intruding a.k.a. primary needle) or a section of the primary needle or the primary needle may come in contact with the region of interest, and insert itself into the myocardium when the heart is enlarged during the cardiac cycle (ventricular diastole) and inject only when such contact is made. The preferred needle-myocardium interaction is orthogonal to minimize any unintended tissue injury during a cardiac cycle. The needle positioning can be adjusted prior, during and after sequential injections by the control system component.

The contact between the injection needle and the myocardium can be monitored via bioimpedance measurement or similar technology. Injection control module of the control system will determine time points for injection, volume to deliver, initiation and stoppage of the injection based on user input and will control delayed/synchronized response/command if necessary to atone for mechanical/electronic delay of the control system.

The injection control is based on the real-time electrocardiogram (ECG) of the patient or study subject. ECG, after amplification and preliminary processing steps, converts the obtained physiological signal into an interpretable waveform, and will be subjected to noise reduction, filtering and thresholding to determine time points at which cyclic diastole begins and ends. Determining the diastolic state or any cardiac phase of interest, alternatively can become possible using another technology such as sonogram. Injection commands will then be sequenced and processed to ensure systematic and automated injections during the diastolic period. Injectable biomaterial/bioactive agents (i.e., the injectate) will be infused via a hydrodynamic system that uses a precision pump 114 in order to control the injectate delivery. In a different embodiment, the injection needle can be equipped with a small chamber to directly deliver injectate during a timepoint dictated by the control system, or the tip of the injection needle can be equipped with a mechanism similar to pump to facilitate delivery.

The term “ventricular diastole” refers to one phase of a biologic activity known as the cardiac cycle, which is one complete heartbeat, during which the ventricles of the heart are relaxed. The heart muscle is divided into four chambers: right atrium, left atrium, right ventricle and left ventricle. Its upper chambers are the two atria, also called auricles, and its two lower chambers are called ventricles. There is much activity involved in one complete cardiac cycle, but it can be divided into two broad phases: diastole and systole.

During ventricular diastole, not only are the ventricles relaxing, they also are filling with blood in preparation for the next phase of the cardiac cycle. There is a significant amount of pressure created in the arteries, and it is necessary for adequate circulation of blood throughout the entire body. Although blood is said to flow throughout the blood vessels of the body, a more accurate description might be that the blood pulsates through the arteries in forceful, rhythmic waves—this creates a pulse that can be felt at certain locations on the body. This is why there are periods of relaxation such as ventricular diastole.

Taking a person's blood pressure is closely related to the subject of ventricular diastole. Blood is forcefully pumped out into circulation with each contraction of the left ventricle, which is a different phase of the cardiac cycle. When the left ventricle is relaxed and refilling with blood, the period of the cardiac cycle known as ventricular diastole is the pressure that is left in the arteries. This pressure is recorded and is called the diastolic blood pressure. The recording of this vital sign itself is an indirect measurement of diastole.

A prototype of the control system (software and hardware) has been developed which performs the following functions: import electrocardiogram (ECG) signal, process the amplified signal and detect left ventricular mid-diastole state (where ventricles are most enlarged during a heart cycle), communicate with a syringe pump to perform injection when the heart wall comes in contact with the injection needle, start material transfer, pause, continue and stop with commands sent from the executable software, command regulating injections, calculate and offset any electromechanical system delay, temporally follow the real-time acquired/simulated ECG signal in an incremental fashion until the predetermined volume has been delivered, and the control system records the injected volume and informs the operator when the treatment has been completed or any errors have occurred during execution of commands.

While there are prior attempts that injectates into the heart, the current disclosure leverages a transepicardial route of administration. For instance, some diseases are present near the epicardium/outer surface of the heart, and hence may be more favorable for physicians to want to access to this area. The present disclosure may be used for various medical pathologies in addition to heart attacks.

Another key differentiation of the present disclosure is that it may or may not move/position the needle at strategic locations to engage with the heart, but the heart is on-beat, no need to use a heart-lung pump to bypass the heart and the heart comes and engages with the needle and that time window is leveraged for injectate delivery.

Further, the current disclosure may introduce/inject a wide variety of materials including but not limited to natural polymers, small molecules, fluids, etc., that have therapeutic potential.

This technology will significantly lower the costs by eliminating the need for invasive surgery. After approval, this procedure will provide faster treatment as well as reduced recovery time for treated patients and can potentially lower intra-operative risk due to invasive surgery. It will also simplify the process for clinicians by outsourcing some detection and infusion steps with use of computers and control systems. Integration of this technology platform into the medical standard of care is expected to have downstream effects on healthcare as well. For example, most heart attack patients are prone to develop heart failure due to adverse remodeling of the heart. Therefore, addressing unmet needs at earlier stages of disease development can reduce burden of heart failures on the healthcare where fewer patients will be good candidates for cardiac repair procedures.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for delivering therapeutic injectates to a heart comprising:
introducing at least one intrusion needle through a chest wall;
positioning the at least one intrusion needle adjacent a heart of a treatment subject via use of at least one cardiac mapping technique;
inserting at least one probing device through the at least one intrusion needle to locate at least one injection site on the heart of the treatment subject;
introducing at least one secondary needle for intrusion into the myocardium; and
injecting a therapeutic injectate via the at least one secondary needle when movement of a heart of the treatment subject causes the heart to engage the at least one secondary needle; and
wherein delivery of the therapeutic agent occurs without performing thoracotomy while the heart is on-beat.

2. The method of claim 1, wherein the at least one probing device is a fiber angioscope producing real time images.

3. The method of claim 1, wherein the at least one probing device is a conductive probe that measures bio-impedance of the myocardium.

4. The method of claim 1, wherein the secondary needle engages the myocardium orthogonally.

5. The method of claim 1, wherein positioning of the secondary needle is adjusted via a control system that can position the secondary needle prior to, during, and after an injection into the myocardium.

6. The method of claim 1, wherein the injection occurs when the heart of the treatment subject is enlarged during the cardiac cycle.

7. The method of claim 1, wherein an injection control module determines time points for injection, volume to deliver, initiation, and stoppage of the injection.

8. The method of claim 7, wherein a hydrodynamic system employing a precision pump controls injectate delivery.

9. The method of claim 1, wherein the at least one injection occurs at an infarct area of the heart.

10. A semi-automated delivery platform for reducing invasive heart operations comprising:
at least one intrusion needle;
at least one control system;
at least one delivery apparatus;
at least one therapeutic agent;
at least one probing device;
at least one injecting device wherein movement of a heart of the treatment subject causes the heart to engage the at least one injecting device; and
wherein delivery of the therapeutic agent occurs without performing thoracotomy and while the heart is on-beat.

11. The semi-automated delivery platform of claim 10, wherein the at least one probing device is a fiber angioscope producing real time images.

12. The semi-automated delivery platform of claim 10, wherein the at least one probing device is a conductive probe that measures bio-impedance of the myocardium.

13. The semi-automated delivery platform of claim 10, further comprising the injecting device engaging the myocardium orthogonally.

14. The semi-automated delivery platform of claim 10, further comprising adjusting positioning of the injecting device via a control system that can position the injecting device prior to, during, and after an injection into the myocardium.

15. The semi-automated delivery platform of claim 10, further comprising timing the injection to occur when the heart of the treatment subject is enlarged during the cardiac cycle.

16. The semi-automated delivery platform of claim 10, further comprising determining via an injection control module time points for injection, volume to deliver, initiation, and/or stoppage of the injection.

17. The semi-automated delivery platform of claim 16, further comprising controlling injectate delivery via a hydrodynamic system employing a precision pump.

18. The semi-automated delivery platform of claim 10, further comprising positioning the at least one injection at an infarct area of the heart.
